(19) United States
(12) Patent Application Publication
(10) Pub. No.: US 2022/0016436 A1
(43) Pub. Date: Jan. 20, 2022

(54) SYSTEM AND METHOD FOR LOW COST LIGHT THERAPY

(71) Applicants: THE GENERAL HOSPITAL CORPORATION, Boston, MA (US); THE UNIVERSITY OF MASSACHUSETTS, Boston, MA (US)

(72) Inventors: Tayyaba Hasan, Arlington, MA (US); Jonathan P. Celli, Boston, MA (US); Imran Rizvi, Chapel Hill, NC (US); Filip Cuckov, Boston, MA (US); Hui Liu, Boston, MA (US)

(21) Appl. No.: 17/292,466

(22) PCT Filed: Nov. 11, 2019
(86) PCT No.: PCT/US19/60723

§ 371 Date: May 10, 2021

Related U.S. Application Data
(60) Provisional application No. 62/758,188, filed on Nov. 9, 2018.
(60) Provisional application No. 62/869,974, filed on Jul. 2, 2019.

(51) Int. Cl. A61N 5/06; A61N 5/067

(52) U.S. Cl. CPC A61N 5/062; A61N 5/0603; A61N 2005/0652; A61N 2005/0606; A61N 2005/063; A61N 5/067

(57) ABSTRACT

A low-cost light delivery system includes a light source device and a light delivery applicator. The light source device includes a light source (e.g., a light emitting diode, a laser, or the like) mounted on a passive heat sink and coupled to an optical fiber extending out of the light source device to carry a light signal. The light source device also includes a microcontroller configured to define a required irradiance to be delivered by the light signal based on a prescribed dosimetry parameter; and one or more power sources, each configured to provide power to at least one of the high powered light emitting diode and the microcontroller. The light delivery applicator can be removably connected to the optical fiber and configured to be placed proximal to an area of the patient to deliver the required irradiance of the light signal to the area of the patient.

### RELATED APPLICATIONS

This application claims the benefit of U.S. Provisional Application No. 62/869,974, entitled “SMARTPHONE-CONTROLLED BATTERY-POWERED PORTABLE LIGHT DELIVERY SYSTEM,” filed Jul. 2, 2019. This application also claims the benefit of U.S. Provisional Application No. 62/758,188, entitled “SMARTPHONE-CONTROLLED BATTERY-POWERED PORTABLE MEDICAL DEVICE FOR INTRAORAL PHOTODYNAMIC THERAPY,” filed Nov. 9, 2018. The entirety of these applications is hereby incorporated by reference for all purposes.

### TECHNICAL FIELD

The present disclosure relates generally to photomedicine and, more particularly, to a system and method for low cost light therapy.

### BACKGROUND

Oral cancer is a major public health problem, particularly in India where it is estimated that over 80,000 new cases of oral cancer are diagnosed per year and one patient dies every six hours. Many of these new cases of oral cancer occur in rural areas of India, where many patients do not receive treatment until the disease has advanced and prognosis is often poor. Even in the most optimal clinical settings, traditional oral cancer treatments, such as surgery and radiotherapy treatment, often present significant side effects, which can impact a patient's ability to chew, swallow, and speak.

### SUMMARY

The present disclosure provides a system and method for low cost light therapy. One example use of the system and method for low cost light therapy includes application of photodynamic therapy within a patient's oral cavity to treat oral cancer.

In one aspect, the present disclosure can include a low cost light therapy system. The system can include a light source device and a light delivery applicator. The light source device includes a light source (e.g., a light emitting diode, a laser, or the like) mounted on a passive heat sink and coupled to an optical fiber extending out of the light source device to carry a light signal generated by the light source. The light source device also includes a microcontroller configured to define a required irradiance based on a prescribed dosimetry parameter; and one or more power sources, each configured to provide power to at least one of the high powered light emitting diode and the microcontroller. The light delivery applicator can be removably connected to the optical fiber and configured to be placed proximal to an area of the patient to deliver the required irradiance of the light signal to the area of the patient.

In yet another aspect, the present disclosure can include a method for providing low cost light therapy. The method includes connecting a light delivery applicator to an optical fiber, wherein the light delivery applicator is configured to be placed proximal to an area of a patient; generating, by a light source mounted on a passive heat sink within a light source device coupled to the optical fiber, a light signal with a required irradiance defined based on a prescribed dosimetry parameter for the area of the patient; and delivering the light signal through the optical fiber and the light delivery applicator to the area of the patient.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a diagram illustrating a system that can be used for low cost light therapy according to an aspect of the present disclosure;

FIG. 2 is a diagram of a light source device shown in FIG. 1;

FIG. 3 is a diagram showing the modular construction of a light delivery applicator shown in FIG. 1;

FIG. 4 shows different example modular components that can be chosen to construct the light delivery device shown in FIG. 1;

FIG. 5 is a process flow diagram illustrating a method for low cost light therapy according to another aspect of the present disclosure;

FIG. 6 is a schematic diagram of an example of a system for low cost light therapy with a light source device, a fiber optic, a light delivery applicator, and a smartphone;

FIG. 7 includes two plots showing the technical performance data for the light source within the light source device;

FIG. 8 shows three example configurations of the light delivery applicator;

FIG. 9 is a photograph showing an example determination of the maximum width of a lesion in a white light image;

FIG. 10 is a radiographic image showing an example determination of the maximum width of a lesion;

FIG. 11 is an image showing an example determination of the maximum width of a lesion in a fluorescence image;

FIG. 12 shows an intraoral light delivery applicator that can provide a certain targeted light diameter;

FIG. 13 is a photograph of a patient receiving intraoral light delivery for photodynamic therapy (PDT) of a lesion; and

FIG. 14 includes a series of photographs tracking the status of the patient's lesion after PDT.

### DETAILED DESCRIPTION

### Definitions

Unless defined otherwise, all technical and scientific terms used herein have the same meaning as is commonly understood by one of skill in the art to which the present disclosure pertains.

In the context of the present disclosure, the singular forms “a,” “an” and “the” can also include the plural forms, unless the context clearly indicates otherwise.

The terms “comprises” and/or “comprising,” as used herein, can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure.

As used herein, the term “photomedicine” can refer to the study and application of light (or phototherapy) with respect to health and disease.

As used herein, the term “photodynamic therapy (PBT)” can refer to a form of phototherapy involving light and a photosensitizing chemical substance (e.g., a drug activated by a certain wavelength of light), used in conjunction with molecular oxygen to elicit cell death.

As used herein, the term “light” can refer to electromagnetic radiation of at least one wavelength provided by a light source. The light source can include one or more light emitting diodes and/or one or more laser sources.

As used herein, the terms “optical fiber” and “fiber optic” can refer to a flexible, transparent fiber made by drawing glass (silica) or plastic to a diameter slightly thicker than that of a human hair used most often as a means to transmit light over a distance between the two ends of the fiber.

As used herein, the term “dosimetry” can refer to the measurement, calculation, and assessment of a dose of light to be absorbed by a patient.

As used herein, the term “irradiance” can refer to optical power per unit area. For example, a light beam configured with a certain irradiance can be configured to deliver a certain power to an area of a patient's body.

As used herein, the terms “area of a patient's body” and “target area” can relate to a diseased or damaged portion of a patient's body in need of medical treatment. In some instances, the target area can be within the patient's oral cavity.

As used herein, the term “oral cavity” refers to the mouth, including the lips, the lining inside the cheeks and lips (buccal mucosa), the front two-thirds of the tongue, the upper and lower gums, the floor of the mouth, the bony roof of the mouth (palate), and the small area behind the wisdom teeth (retromolar).

As used herein, the term “substantially” can refer to a majority of something being in a condition. In some instances, the majority can be 50% or more. In other instances, the majority can be 55, 60, 65, 70, 75, 89, 85, 90, 95, 97, or 99% or more.

As used herein, the terms “subject” and “patient” can be used interchangeably and refer to any warm-blooded organism including, but not limited to, a human being, a pig, a rat, a mouse, a dog, a cat, a goat, a sheep, a horse, a monkey, an ape, a rabbit, a cow, etc.

Overview

The present disclosure relates to a system and method for low cost light therapy, including a light source device (including a light source coupled to an optical fiber, which provides a flexibility for light delivery in a semi-enclosed space) and a light delivery applicator (removably connected to the optical fiber and configured to be placed proximal to an area of the patient to deliver a required irradiance of the light signal to an area of the patient). The light delivery applicator can be sized, dimensioned, and/or oriented to deliver the required irradiance to the area of the patient. In some instances, a portable computing device can provide an interface for dosimetry calculations and control/feedback via a wireless connection (e.g., Bluetooth). The system and method can be used, for example, for photodynamic therapy (PDT), cosmetic applications, pain relief, wound healing, medical research (e.g., cancer biology research), and/or other photomedicine applications.

The optical fiber of the light source device used with the specific light delivery applicator can address a significant technological unmet need that has stymied the broader clinical implementation of PDT for treatment of oral lesions. While early clinical studies of PDT for treatment of oral lesions have all had generally positive results, showing effective tumor destruction and outstanding healing of the mucosa, the approach has never been widely adopted. Specifically, the PDT dosimetry parameters, which require light delivery for intervals of 10 to 30 minutes, manual free space light delivery (a clinician holding a fiber optic and pointing it into a patient's mouth) is prohibitively cumbersome. Accordingly there is an unmet need for robust, simple-to-use technology, which the system and method of the present disclosure addresses with the light delivery applicator, which has a modular design that can be customizable for different lesion sizes, lesion position (retromolar, posterior/anterior buccal, palate, etc.), as well as mouth/jaw size. Additionally, the light delivery applicator can simplify the potentially complex dosimetry calculations by providing contact mode light delivery. Since the average irradiance over the applicator surface is pre-calibrated, it is not necessary for the user to measure the spot size and calculate the power over the area of the beam spot, which in general is difficult to control with a typical handheld light delivery implementation.

Systems

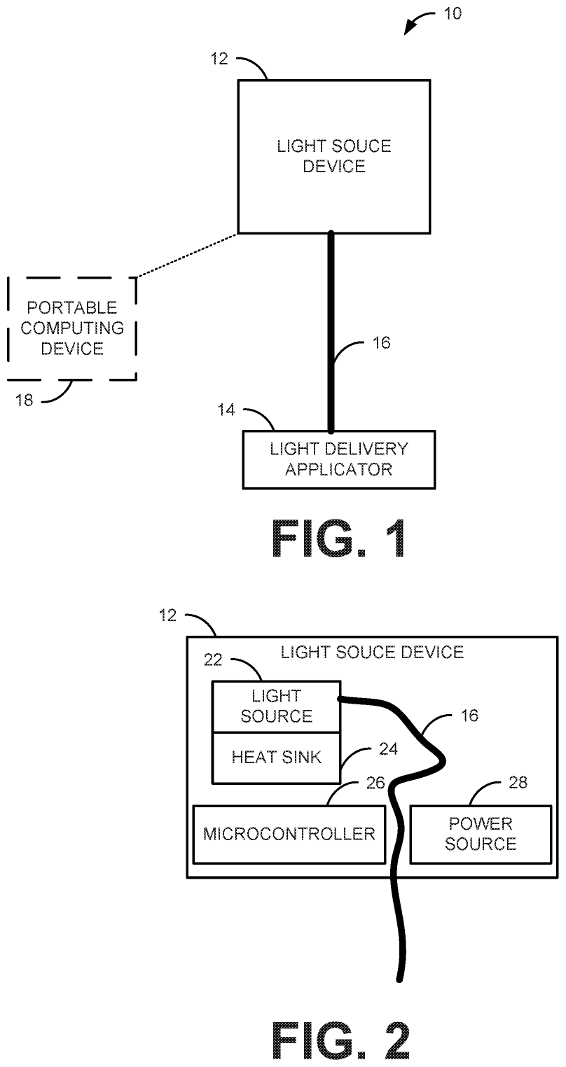

FIG. 1 illustrates a system 10 that can be used for low cost light therapy. The system 10 can be used, for example, for photodynamic therapy (PDT), cosmetic applications, pain relief, wound healing, medical research (e.g., cancer biology research), and/or other photomedicine applications. For ease of description, the system 10 will be described with regard to intraoral light delivery for photodynamic therapy (PDT), but the system 10 is not limited to intraoral light delivery for PDT.

In its simplest form, the system 10 includes a light source device 12 and a light delivery applicator 14 (or “light delivery applicator”). An optical fiber 16 (also referred to as a fiber optic cable), at least a portion of which may be non-rigid, extends from the light source device 12 to the light delivery applicator 14. In some instances, the light source device 12 can be sized, shaped, and/or weighted so that the light source device 12 is portable.

As shown in FIG. 2, the optical fiber 16 extends from a light source 22 within the light source device 12, out of the light source device 12, and to the light delivery applicator 14. Light can be transmitted from the light source 22, through the optical fiber 16, through the light delivery applicator 14, which is specifically designed to deliver the light to a target area within a patient's body according to a precalibrated dosimetry based on a property of the target area and/or the light delivery applicator. The light delivery applicator 14 can be removably coupled to the optical fiber 16 and configured to be placed proximal to an area of the patient to deliver the required irradiance of the light signal to the area of the patient according to the dosimetry. In some instances, the light source device 12 can be in wireless communication with a portable computing device (e.g., a smartphone, a tablet, a laptop, etc.), which can be used to define properties of the light source device 12.

As shown in FIG. 2, the light source device 12 can include a light source 22 (e.g., one or more light emitting diodes, high powered light emitting diodes, lasers, etc.) that can be configured to generate light at an optical power. The light generated by the light source device 12 can include one or more wavelengths. For example, the one or more wavelengths can be between 600 and 700 nm. In some instances, the light source 22 can be mounted on a passive heat sink 24. The light source 22 can be coupled to the optical fiber 16 within the light source device 12. In some instances, at least a portion of the optical fiber 16 can be non-rigid.

The light source device 12 can also include a microcontroller 26 that can control the delivery of light by the light source 22. For example, the microcontroller 26 can be configured to define how the light is generated based on a required irradiance to be delivered based on a prescribed dosimetry parameter. The microcontroller 26 can control circuit elements within the light source device 12 (e.g., to provide a certain current output). The microcontroller 26 can also provide a user interface to provide visualization and/or control of operating procedures of the light source device 12, the light source 22, or other components of the light source device 12. As an example, the user interface can display outputs regarding a treatment procedure and receive inputs related to the treatment procedure.

The light source device 12 can also include one or more power sources 28. The one or more power sources 28 can each be configured to provide power to at least one of the light source 22 and the microcontroller 26. As an example, the one or more power sources can include a rechargeable battery and/or a direct current (DC) applicator. The light source device 12 can include one or more additional components, including (for example) a wireless transmitter (e.g., a Bluetooth transmitter) configured for transmission in at least a local area. The wireless transmission can be unidirectional and/or bidirectional with a portable (or mobile) computing device 18, which can control one or more settings of the light source device 12 (e.g., the settings can be controlled by the microcontroller 26). As another example, the additional components can include circuit components associated with the light source 22 and/or the microcontroller 26.

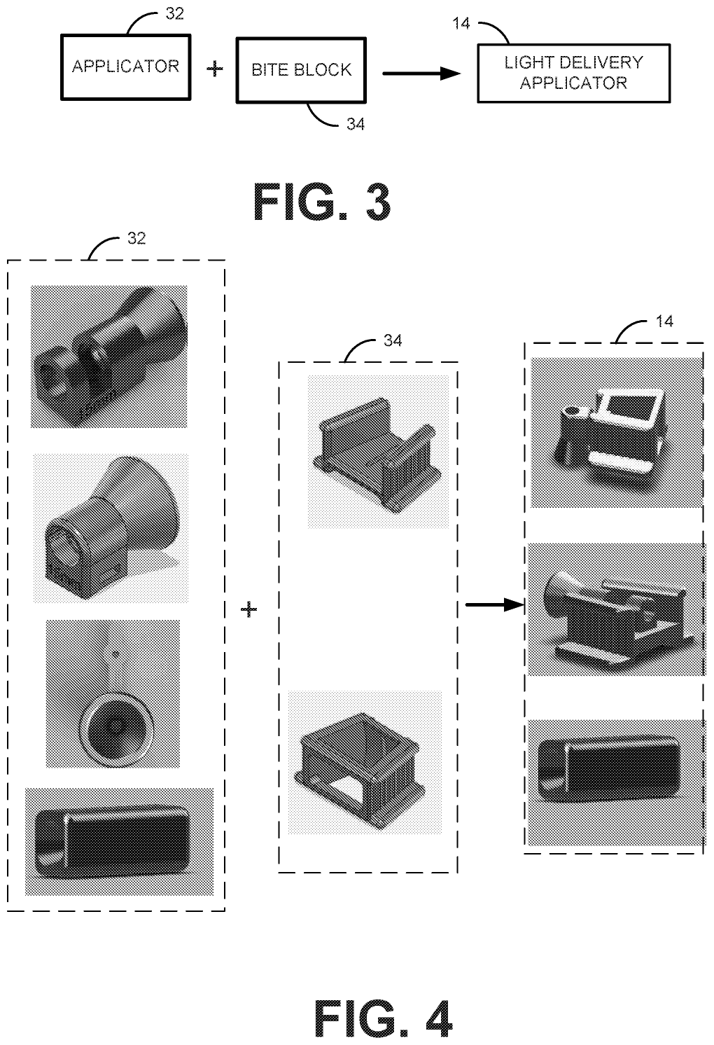

The optical fiber 16 can receive the light and transmit a light signal out of the light source device 12, eventually to the light delivery applicator 14. The light delivery applicator can apply the light signal to the target area based on a controlled dosimetry. Example configuration of the light delivery applicator 14 are shown in FIGS. 3 and 4. The light delivery applicator 14 can be removably connectable to the optical fiber and configured to be placed proximal to (e.g., directly against or near) an area of the patient to deliver the required irradiance of the light signal to the area of the patient, as shown in FIG. 3. A different size, shape, configuration, etc. of the light delivery applicator can be chosen based on a size, depth, location, etc. of a target area within the patient's body.

In one example, the target area can include a lesion within the oral cavity of a patient (also referred to as “the mouth”). The light delivery applicator 14 can provide robust ergonomic intraoral light delivery with controlled dosimetry to be delivered to a specific spot size. Notably, the light delivery applicator 14 can be customized for the patient based on a size of the mouth, a jaw size, a position of a lesion, a size of the lesion, or the like, in such instances, the light delivery applicator 14 can act as a spacer that fixes the optical fiber 16 away from the tissue within the mouth of the patient that the divergent beam from the optical fiber expands to the calibrated spot size of the target area.

FIG. 3 illustrates an example where the light delivery applicator 14 includes two separate modular pieces—an applicator 32 and a mouth prop 34. The applicator 32 and the mouth prop 34 can be selected based on a size of the mouth, a jaw size, a position of a lesion, a size of the lesion, or the like, meaning that different sized and shaped light delivery applicators 14 are possible. The applicator 32 can be selected to direct light to the lesion. In fact, the applicator 32 can be chosen from a plurality of applicators (examples shown in box 32 of FIG. 4). The mouth prop 34 can be configured to orient the applicator 32 and/or the optical fiber 16 in a correct position within the oral cavity of the patient, selected based a plurality of mouth guards (examples shown in box 34 of FIG. 4; in some instances, no mouth guard is selected). The modular components can be combined into the light delivery applicator 14 (examples shown in box 14 of FIG. 4).

In some instances, the applicator 32 and/or the mouth prop 34 can be 3D-printed according to a specification customized for the particular patient or chosen from a plurality of specifications based on one or more properties of the target area. The 3D-printing can enable one or more modules (the applicator 32 and/or the mouth prop 34) of the light delivery applicator 14 to be interchangeable based on the location of the target area and/or the size of the target area. In other instances, the applicator 32 and the mouth prop 34 can be a single device.

In some instances, the portable computing device 18 can provide feedback and control for the system 10. The portable computing device 18 can include a non-transitory memory storing instructions and a processor to execute the instructions. For example, the memory can store an application that can be executed by the processor to determine dosimetry properties. For example, the dosimetry properties can be determined based on one or more user inputs for the optical output at the tip of the optical fiber, the applicator selection, the treatment duration, the recommended fractionation (time intervals for breaks in light delivery). As another example, the memory can include instructions that are executed by the processor to control one or more properties that are controlled by the microcontroller. As a further example, the memory can include instructions that are executed by the processor to record images of the target area to track the progress of treatment of the target area and/or determine boundaries of the target area. The imaging can be fluorescence imaging using an attachment to the portable computing device 18.

### Methods

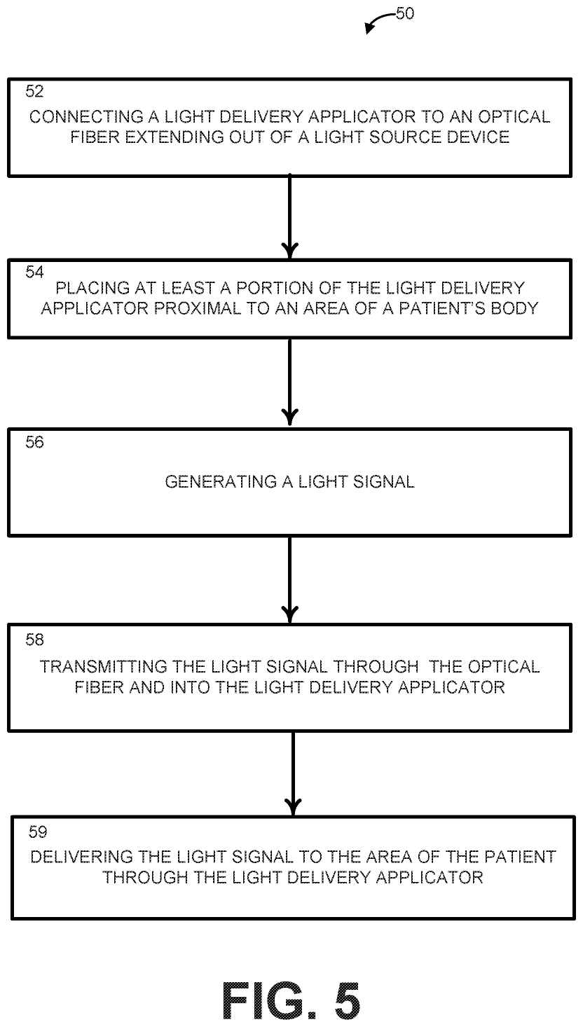

Another aspect of the present disclosure can include a method 50 (FIG. 5) or low cost light therapy. The method 50 can be performed, for example, by the system of FIG. 1 using components shown in FIGS. 2-4. The method 50 can be used for example, for photodynamic therapy (PDT), cosmetic applications, pain relief, wound healing, medical research (e.g., cancer biology research), and/or other photomedicine applications. For example, one use of the method 50 can be for intraoral light delivery for photodynamic therapy (PDT) to treat lesions within the oral cavity.

The method 50 is illustrated as a process flow diagram with flowchart illustrations. For purposes of simplicity, the method 50 is shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the method 50.

At 52, a light delivery applicator (e.g., light delivery applicator 14) can be connected to an optical fiber (e.g., optical fiber 16) extending out of a light source device (e.g., light source device 12). At 54, at least a portion of the light delivery applicator (e.g., the applicator 32 of light delivery applicator 14) can be placed proximal to an area (e.g., a target area) of a patient's body. In some instances, the portion of the light delivery applicator can be directly contacting the area.

At 56, a light signal can be generated (e.g., by a light source 22 within the light source device 12). The generation of the light signal can be based at least in part on dosimetry parameters, which in some instances can be determined by a portable computing device (e.g., portable computing device 18). In some instances, the dosimetry and/or other control parameters can be input on a user interface of the light source device which can also display outputs regarding the treatment procedure. At 58, the light signal (e.g., from the light source 22 within the light source device 12) can be transmitted through the optical fiber into the portion of the light delivery applicator (e.g., the applicator 32 of light delivery applicator 14).

At 59, the light signal can be delivered to the area of the patient through the portion of the light delivery applicator (e.g., the applicator 32 of light delivery applicator 14). After the required irradiance is delivered (e.g., according to the dosimetry profile determined at least in part by the configuration of the applicator 32 of the light delivery applicator 14), at least a portion of the light delivery applicator (e.g., light delivery applicator 14) can be removed from the optical fiber (e.g., optical fiber 16) so that the optical fiber can be reused.

### Example—Intraoral Photodynamic Therapy (PDT)

### Configuration of Devices

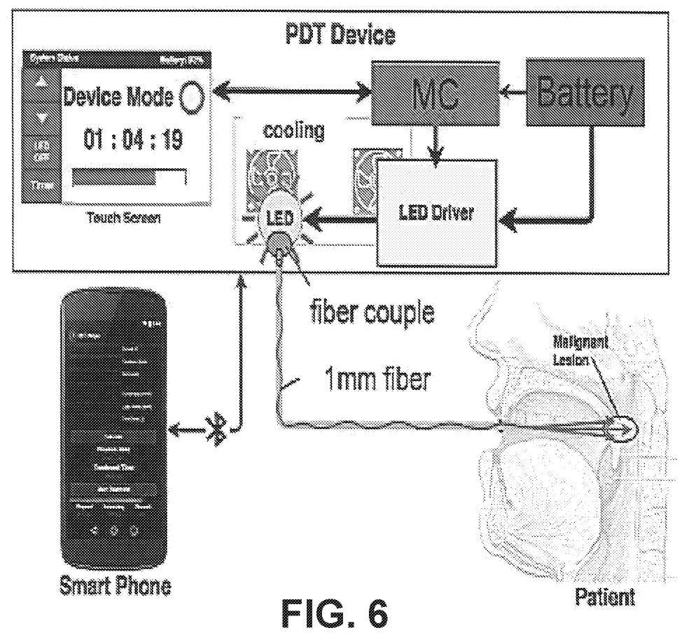

The system 10 and method 50 described herein were designed for use in connection with intraoral PDT. Specifically, PDT using d-aminoleyulinic acid (ALA) photosensitization was chosen because fewer potential side effects were seen and because light delivery can be carried out with limited medical infrastructure. FIG. 6 shows the general setup of a PDT device (an implementation of the light source device 12) with a 1 mm optical fiber coupled to the LED source. The optical fiber is also coupled outside the PDT device to the light delivery applicator, which is designed and chosen to shine light at a malignant lesion within a patient's oral cavity. A smart phone can control aspects of the PDT device.

The PDT device can be housed in a 14 cm×16 cm×12 cm enclosure weighing a total of about 1 pound. Inside the enclosure, a high power 635 nm light emitting diode can be mounted on a passive heat sink and coupled to a 1 mm diameter multimode optical fiber to separate the heat/electronics if the light emitting diode and associated circuitry and the location where the light signal is delivered. The total optical power at the distal end of the fiber is approximately 110 mW, which is distributed over the tissue surface via means of an interchangeable light delivery applicator that attaches to the end of the fiber. The internal LED can be powered using a voltage regulator configured to provide constant current output. An array of relays under digital control switch the output of the voltage regulator through a network of resistors, which change the output current driving the LED. A separate voltage regulator can provide power to a commercial microcontroller, which controls the relays and provides a user interface (UI). The UI can display the current power setting and manage inputs that switch the optical power level and enable/disable the LED (should treatment need to be paused). In addition to the UI on the front panel of the device itself, the microcontroller also includes embedded wireless communication via Bluetooth, allowing control of the device's setting from a smartphone app. The light source operates on battery power, using commercial rechargeable 7.4 V lithium polymer battery, or a DC adapter which connects in the back. The microcontroller monitors the battery voltage via an on-board analog-to-digital converter and warns the user via a display as well as any connected mobile device when the battery is running low.

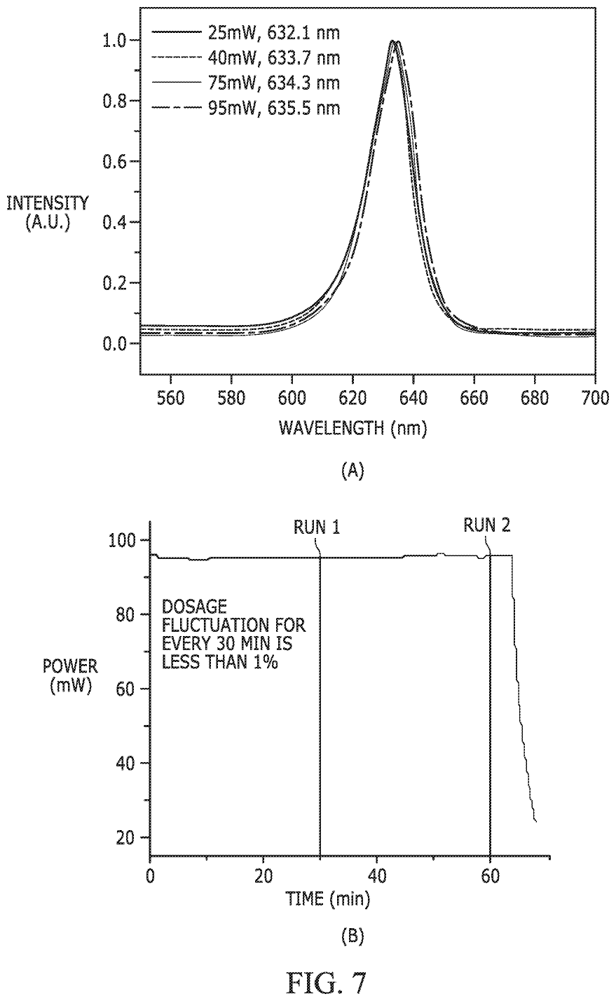

FIG. 7 shows the technical performance data for the LED-based PDT light source (spectral characteristics and optical power output for PDT). Spectral characteristics (a) shows a peak wavelength of approximately 632-635 nm, within the window that is considered optimal for therapeutic activation of protoporphyrin IX, which is induced by ALA photosensitization. The battery-powered device operation using 7.4 V lithium polymer batteries achieves constant total power with minimal fluctuation for 2 consecutive 30 minute runs mimicking typical PDT procedure duration (b).

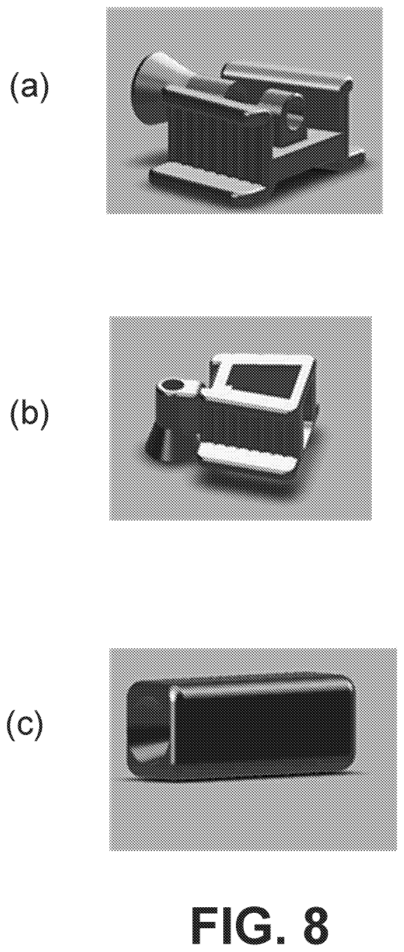

Example light delivery apparatuses are shown in FIG. 8. These devices address the need for robust ergonomic intraoral light delivery with controlled dosimetry through a system of interchangeable 3D-printed pieces for controlled illumination of lesions on the retromolar, palate, anterior or posterior buccal regions with spot sizes of 1, 1.5 or 2 cm in diameter. This system of modular component includes “applicators” and “mouth props.” The applicators attach directly to the fiber at one end and contact the tissue surface at the other, acting as a spacer that fixes the fiber tip back sufficiently far from the tissue surface that the divergent beam from the fiber expands to the calibrated spot size on the tissue surface. The appropriate applicator for a lesion of a given size can be mounted to a mouth prop which orients the fiber/applicator to the correct position within the oral cavity (see (a) and (b)). For certain locations (upper palate and retromolar) the applicator and mouth prop is a single assembly (e.g., (c)). This contact mode light delivery greatly simplifies dosimetry since the PDT dose is determined directly by the irradiance (optical power per unit area) at the tissue surface. In traditional free space delivery, unless perfect collimation is achieved, the spot size (and hence the area over which total power is divided) is strongly dependent on the distance from the fiber optic to the tissue surface, a parameter which is difficult to control. The contact mode delivery of the light delivery apparatuses mitigate the downfalls of traditional free space delivery.

The applicators and mouth props can be 3D-printed on demand to patient-customized specifications, in some instances. Since computer aided design files can be rapidly updated to specific dimensions based on an individual patient's lesion size, range of jaw motion, dental conditions, etc., customized modular components can be created rapidly by a 3D-printer. In other instances, the applicators and mouth props can be chosen from a set of pre-existing devices according to lesion shape and location.

A smartphone-based device can be used for feedback and control of the PDT device. To simplify user operation, PDT dosimetry calculations can all be performed using a “PDT helper” App developed for Android OS. Based on user inputs for the optical output at the fiber tip and the applicator selection treatment duration and recommended fractionation (time intervals for breaks in light delivery) can be automatically calculated. Furthermore, protoporphyrin IX tumor fluorescence can also be captured using a smartphone camera in combination with a simple modification of a commercially available blue/violet LED array that clips around the smartphone camera. It is envisioned that the smartphone App will use this fluorescence image data to automatically determine the lesion boundaries (as we and others routinely do in off-line image processing) to inform the applicator size selection and complete the dosimetry calculation based on only the measured total power from the optical fiber.

### Experimental Methods and Results

The PDT therapy with the low cost, minimally invasive set-up described above was used for patients in India with oral lesions. Oral cancer is a health crisis in India, with 30% of all cancers being oral cancer which may be due to the widespread chewing of tobacco, betel nut, and acacia extract. Surgery and radiotherapy are not readily available to many patients at rural sites. Even where possible, surgery and radiotherapy may be potentially disfiguring. There is an urgent need for effective, yet low cost, treatment and imaging without need for major medical infrastructure, so a low cost, rugged/portable, battery operated system with potential telemedicine integration, as described above would be especially useful in India.

Patients with proven biopsy having T1N0M0 malignant lesions in the Buccal Mucosa with normal biochemical parameters and no co-morbid illnesses were selected. A total of 21 patients (19 male, 2 female, from 24-64 years old with a median of 42 years old) with 24 lesions were selected. Post PDT therapy, 14 patients with 17 tumors exhibited a negative biopsy, but 6 patients with 6 tumors showed signs of residual disease. 1 patient was lost to follow up. Better results from the PDT therapy were seen with a modified differentiated SCC compared to a well differentiated SCC.

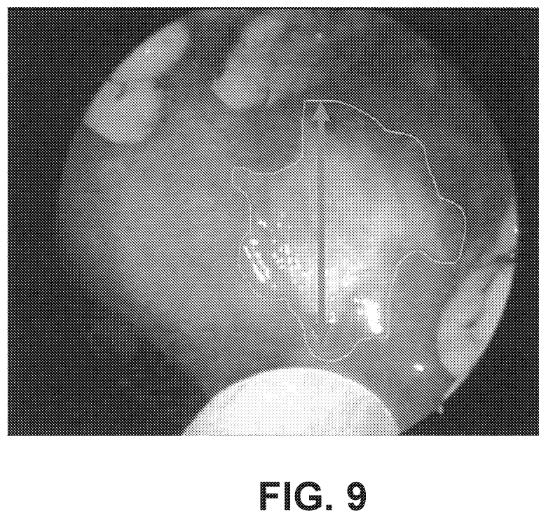

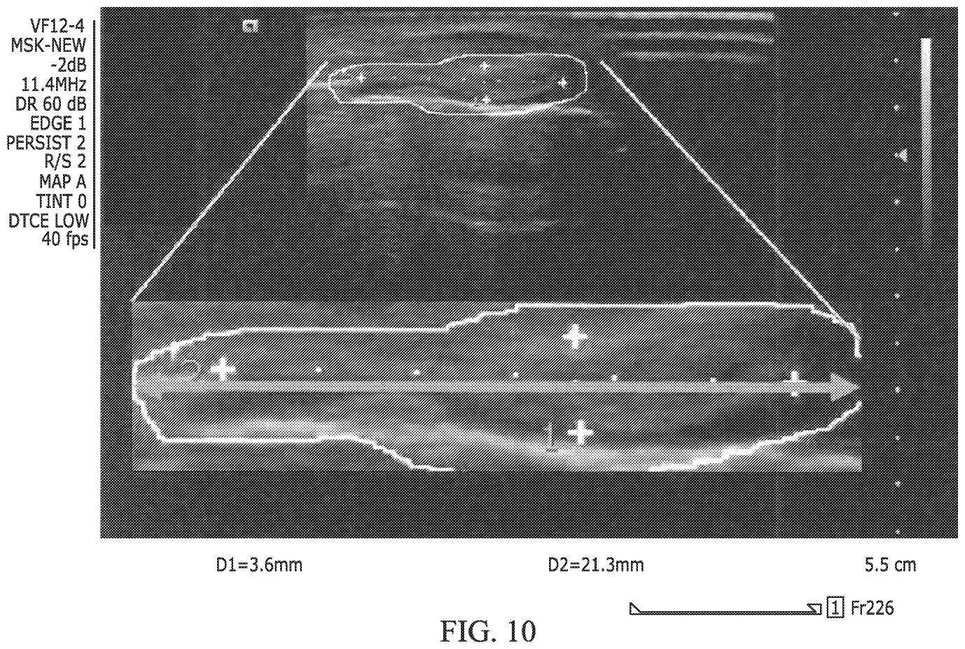

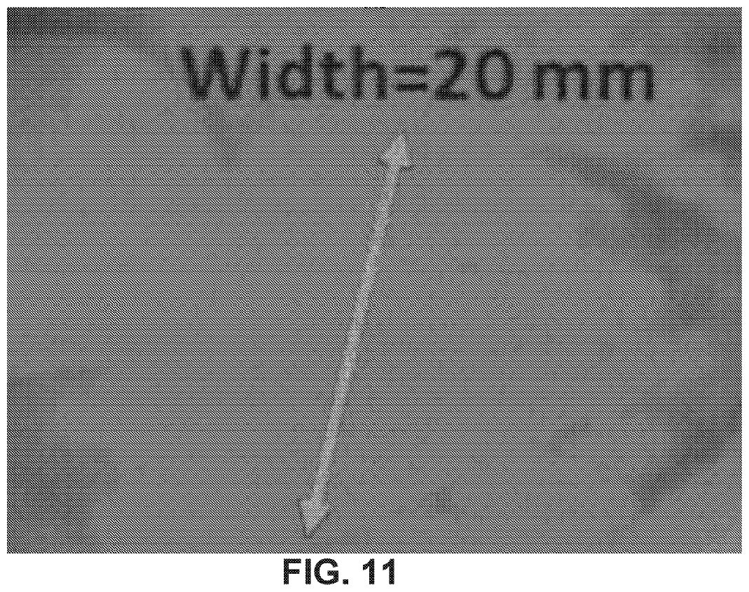

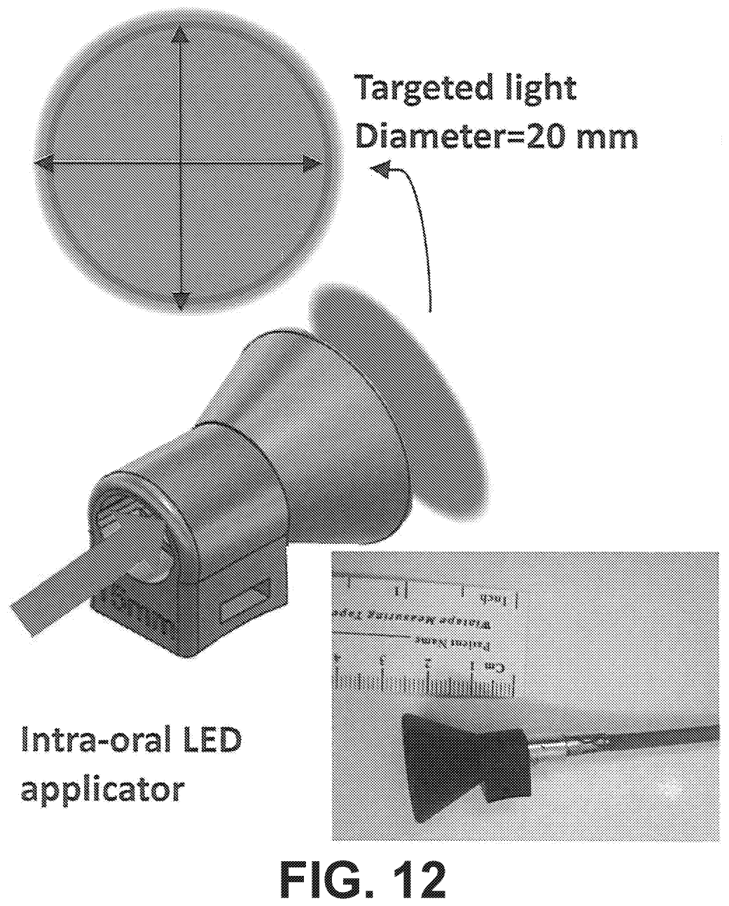

For each patient, a lesion size was determined and an applicator size was chosen. The lesion size was determined using fluorescence imaging (FIG. 11, 20 mm max width), which was determined to be accurate compared to white light imaging (FIG. 9, 19 mm max width) and radiographic imaging (FIG. 10, 21 mm max width). Dosimetry parameters were calculated based on the applicator and the lesion size and input into the PDT device. The applicator of FIG. 12 is configured to generate a spot of a certain targeted light diameter (in this example, the lesion size was determined to be 20 mm, the applicator was chosen to provide a target light diameter of 20 mm).

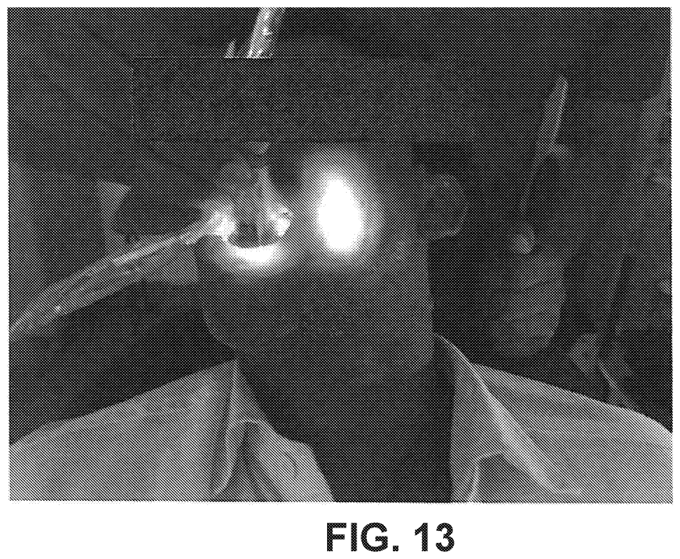

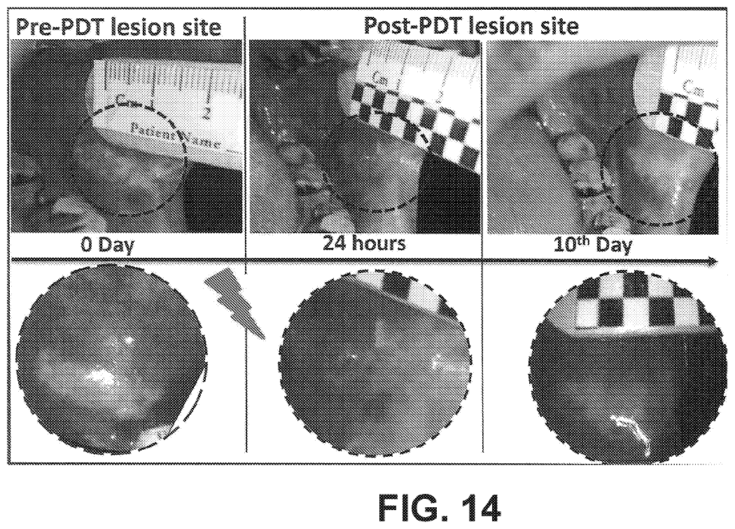

The LED light source delivered a light signal of 630 nm and a power density of 50 mW/cm² according to the dosimetry. The fractionated light treatment dose was 100 J/cm² in fractions of 10 minutes each with 2 minutes interfraction intervals. The application of the light tot the patient is shown in FIG. 13. FIG. 14 shows the temporal progress of the treatment procedure with the intraoral PDT. Treatment outcomes were comparable to conventional treatment methods with faster recovery, safe, effective, and repeatable with no complications. Set up was achievable in remote/rural areas with less infrastructure and skill.

From the above description, those skilled in the art will perceive improvements, changes and modifications. Such improvements, changes, and modifications are within the skill of one in the art and are intended to be covered by the appended claims.

What is claimed is:

1. A system comprising:
a light source device comprising:
a light source mounted on a passive heat sink and coupled to an optical fiber extending out of the light source device, wherein the optical fiber delivers a light signal with a predefined optical power out of the light source device;
a microcontroller configured to define a required irradiance to be delivered by the light signal based on a prescribed dosimetry parameter; and
one or more power sources, each configured to provide power to at least one of the light source and the microcontroller; and
a light delivery applicator removably connected to the optical fiber and configured to be placed proximal to an area of the patient to deliver the required irradiance of the light signal to the area of the patient.

2. The system of claim 1, further comprising a portable computing device configured to wirelessly communicate with the light source device,
wherein the portable computing device is configured to define a size of the area of the patient and the prescribed dosimetry to be delivered by the light signal and sends the prescribed dosimetry parameter to the microcontroller.

3. The system of claim 2, wherein the portable computing device is configured to image the area of the patient to facilitate monitoring and/or guiding of treatment provided by the required irradiance.

4. The system of claim 1, wherein the light delivery applicator is configured to deliver the required irradiance to an area of a mouth of the patient, and
wherein the light delivery applicator is customized for the patient based on at least one of a size of the mouth, a jaw size, a position of a lesion, and a size of the lesion.

5. The system of claim 4, wherein the light delivery applicator comprises:
an applicator configured to attach to the optical fiber at one end and contact tissue at another end acting as a spacer that fixes the optical fiber away from tissue within the mouth of the patient.

6. The system of claim 5, wherein light deliver applicator further comprises a mouth prop configured to orient the applicator and/or the optical fiber in a correct position within the mouth of the patient.

7. The system of claim 6, wherein the applicator and/or the mouth prop are 3D-printed according to a specification customized for the patient.

8. The system of claim 1, wherein the one or more power sources comprises a rechargeable battery or a direct current (DC) adaptor.

9. The system of claim 1, wherein the light source device further comprises a user interface to display outputs regarding a treatment procedure and receive inputs related to the treatment procedure.

10. The system of claim 1, wherein the light source device is portable.

11. The system of claim 1, wherein the light source comprises a high powered light emitting diode configured to provide a light with a wavelength specific for therapeutic activation of a chemical used to treat a medical condition within the area of the patient.

12. The system of claim 11, wherein the wavelength is between 600 nm and 700 nm.

13. A method comprising:
connecting a light delivery applicator to an optical fiber, wherein a portion of the light delivery applicator is configured to be placed proximal to an area of a patient;
generating, by a light source within a light source device coupled to the optical fiber, a light signal; and
delivering the light signal through the optical fiber and portion of the light delivery applicator to the area of the patient.

14. The method of claim 13, wherein the area of the patient is an area of a mouth of the patient, and
wherein the light delivery applicator is customized for the patient based on at least one of a size of the mouth, a jaw size, a position of a lesion, and a size of the lesion.

15. The method of claim 14, wherein the light delivery applicator comprises an applicator configured to attach to the optical fiber at one end and contact tissue at another end acting as a spacer that fixes the optical fiber away from tissue within the mouth of the patient.

16. The method of claim 15, wherein light deliver applicator further comprises a mouth prop configured to orient the applicator and/or the optical fiber in a correct position within an oral cavity within the mouth of the patient.

17. The method of claim 13, further comprising receiving, by the light source device, the prescribed dosimetry from a portable computing device.

18. The method of claim 13, further comprising at least one of displaying, on a user interface associated with the light source device, outputs regarding, by the user interface associated with the light source device, a treatment procedure and receive inputs related to the treatment procedure

19. The method of claim 13, wherein the optical fiber extends from the light source within the light source device to the light delivery applicator, thereby preventing heat associated with of the light source device from touching tissue associated with the area of the patient.

20. The method of claim 13, further comprising removing the light delivery applicator from the optical fiber when the required irradiance is delivered by the light signal.
